(19) United States
(12) Patent Application Publication
(10) Pub. No.: US 2009/0311028 A1
(43) Pub. Date: Dec. 17, 2009

(54) LIQUID DISPENSER FOR MEDICAL PURPOSES IN THE FORM OF AN APPLICATOR AND USE OF THE SAME

(76) Inventors: Erich Odermatt, Schaffhausen (CH); Gabriel Siedle, Freiburg (DE)

Correspondence Address: IP GROUP OF DLA PIPER LLP (US), ONE LIBERTY PLACE, 1650 MARKET ST, SUITE 4900, PHILADELPHIA, PA, 19103, US

(21) Appl. No.: 12/374,061

(22) PCT Filed: Jul. 13, 2007
(86) PCT No.: PCT/EP2007/006225

§ 371 Date: Feb. 27, 2009

(30) Foreign Application Priority Data
Jul. 18, 2006 (DE) 10 2006 034 272.0

(51) Int. Cl. B05C 21/00; A61B 17/03

(52) U.S. Cl. 401/132; 606/214; 401/196

(57) ABSTRACT

A liquid dispenser for medical purposes in the form of an applicator, having an outer container, which comprises a hollow application aid made from a nonporous material, in the form of a cannula, and having an inner, vaportight, and closed container with a curable liquid located therein, the application aid having a delivery opening with a clear cross section of between about 0.1 and about 5 mm².

### RELATED APPLICATIONS

This is a §371 of International Application No. PCT/EP2007/006225, with an international filing date of Jul. 13, 2007 (WO 2008/009386 A1, published Jan. 24, 2008), which is based on German Patent Application No. 102006034272.0, filed Jul. 18, 2006.

### TECHNICAL FIELD

This disclosure relates to a liquid dispenser for medical purposes, in the form of an applicator, and also to its use in medicine.

### BACKGROUND

Modern-day medicine presently has a multiplicity of different treatment possibilities available for wound treatment, in particular for wound closure. Routinely, for example, suture materials and what are called surgical tacking devices are employed. These can be used to successively treat large and, in particular, deep wounds. However, this form of wound treatment causes a certain degree of traumatization of the wound environment. A further disadvantage is the formation of scar tissue, which especially in the case of wounds treated superficially can lead to cosmetically unsatisfactory results.

As an alternative to the above-described wound closure materials, therefore, liquid adhesive compositions as well are increasingly being used in wound management. Such adhesive compositions are based in particular on cyanoacrylate monomers. One such composition, for example, is sold commercially under the name HISTOACRYL® by B. Braun Melsungen AG.

The cyanoacrylate monomer comprises monomers which cure rapidly in the presence of bodily fluids. Adhesive compositions based thereon must therefore be stored and kept in a form which prevents premature curing of the adhesive composition. At the same time, the adhesive compositions must be applied in a sterile state to the wound, to rule out possible wound infections. Corresponding applicator systems for administering the adhesive compositions must meet these requirements. Moreover, it ought to be possible to produce applicator systems of this kind at a cost which as far as possible is acceptable economically.

Before being applied, adhesive compositions are frequently kept or stored in glass ampules. Within the glass ampules, the adhesive compositions are indeed protected from moisture and atmospheric oxygen. However, glass ampules have proven largely to be awkward in terms of their handling in the practice of medical treatment. The ampules must first be broken to release the adhesive compositions. On the one hand, this makes controlled application of the compositions more difficult, and on the other hand it poses a certain risk of injury to the surgeon from the broken glass.

EP 0 832 137 B1 discloses an applicator for an adhesive composition, comprising an inner glass ampule, which contains an adhesive composition and an outer plastic ampule. Exerting an external pressure on the plastic ampule causes the inner glass ampule to fracture, and, consequently, the adhesive composition emerges from the glass ampule and can be applied to a wound via a discharge opening. To prevent glass splinters entering the wound, the discharge opening has a fine porosity. A disadvantage there, however, is that, owing to the porosity and size of the discharge opening, the adhesive composition may also emerge at the periphery of the discharge opening. In this way, controlled release of the adhesive composition from the applicator is made more difficult.

One development of an applicator system of that kind is known from WO 01/51218 A1, whereby the discharge opening may be provided with furrows or channels, which in particular are porous. They are intended to allow focused application of the adhesive compositions. Depending on the configuration of the discharge opening, applicator systems of this kind can be produced only with a relatively high level of technical complexity.

It could, therefore, be helpful to provide an economically advantageous applicator system for adhesive compositions which on the one hand meets the ever-growing requirements in terms of the sterility and the storage stability of adhesive compositions, and on the other hand allows extremely simple and low-risk handling in everyday medical treatment practice.

### SUMMARY

We provide a liquid dispenser for medical purposes in the form of an applicator, having an outer container, which includes a hollow application aid made from a nonporous material, in the form of a cannula, and having an inner, vaportight, and closed container with a curable liquid located therein, the application aid having a delivery opening with a clear cross section of between about 0.1 and about 5 mm².

We also provide a method of treating human and/or animal wounds including contacting a liquid dispensed from the liquid dispenser with a human and/or animal wound.

### BRIEF DESCRIPTION OF THE DRAWINGS

The figures show the following schematically (not to scale):

FIG. 1: liquid dispenser with polyurethane foam;

FIG. 2: liquid dispenser with shrink-on polyethylene tube;

FIG. 3: liquid dispenser with aluminum pouch; and

FIG. 4: liquid dispenser in a pack with a sterilizing window integrated at one end.

### DETAILED DESCRIPTION

We provide a liquid dispenser for medical purposes in the form of an applicator, having an outer container, which comprises a hollow application aid made from a nonporous material, in the form of a cannula, and having an inner, vaportight, and closed container with a curable liquid located therein, the application aid having adelivery opening with a clear cross section of between about 0.1 and about 5 mm².

We also provide an applicator system which, in particular, allows controlled and focused application of the curable liquid to a wound to be treated, without the liquid prematurely curing and/or without the wound environment coming into unwanted contact with other components of the applicator system.

The delivery opening of the liquid dispenser can be designed in different forms. For example, the delivery opening may be a nozzle, more particularly a slotted nozzle. The application aid is advantageously of cylindrical or conical design. The application aid is preferably designed as an application tip, in particular in the manner of a cannula or pipette tip, preferably in the manner of a cannula.

The delivery opening may be open or closed. For example, the delivery opening can be opened only shortly before the liquid is discharged, by means, for example, of the closing end of the application aid being cut off. In general, the delivery opening of the application aid is opened. In this way it is possible, with particular advantage, for sterilization of the interior of the outer container to take place, in particular the outside of the inner container, of the liquid dispenser of the invention.

In one preferred construction the delivery opening has a clear cross section of between about 0.2 and about 3.0 mm², in particular between about 0.5 and about 2.0 mm², in particular between about 0.5 and about 1.0 mm², preferably of about about 0.8 mm². Delivery openings having clear cross sections of this kind permit accurate and, in particular, directed application of the liquid to wound areas. This is particularly advantageous in the context in particular of the treatment of relatively small wounds. The cross section of the opening can be shaped in a desired way—for example, circularly, ovally or flatly.

In a further construction, located between the inner container and the delivery opening of the application aid, there is a porous barrier which is pervious for the curable liquid and impervious for fragments of the inner container. In this way, for the purpose of administering the liquid, the inner container can be destroyed, by means, for example, of exertion of pressure on the outer container, with constituents of the inner container being retained by the barrier.

In a further construction, the barrier possesses pores having a diameter of between about 0.1 and about 500 μm, preferably between about 10 and about 200 μm. The barrier is preferably designed as a porous membrane, in particular in the form of a filter. Provision may likewise be made for the barrier to be designed as a sponge or foam.

On its inner surface, the application aid of the liquid dispenser preferably has fastening means for the porous barrier. With particular preference the fastening means are bulges in the inner surface of the application aid. Adhesive bonding, welding or clipping techniques are suitable for the fastening of the barrier.

Provision may further be made for the porous barrier itself to have a solid support. The support may be, for example, a perforated disc on which the barrier is applied. Particularly suitable materials for the support include metals or plastics. The barrier may be joined to the support by adhesive bonding or welding techniques. The barrier is preferably applied on the support. The support serves advantageously to stabilize the barrier. In this way it is possible to prevent the barrier springing out or being damaged in the course of use of the liquid dispenser.

In another construction the porous barrier envelops the inner container, preferably completely. The porous barrier is preferably designed as a liquid-pervious envelope for the inner container. The barrier may, for example, be a foam cladding. It is also possible for the barrier to be a liquid-pervious film, which in particular may have perforations. In one preferred construction the porous barrier is designed as a liquid-permeable flexible tube, a woolen tube, for example. With particular preference the barrier is designed as a liquid-pervious shrink-on tube. In this way, after being filled with the curable liquid, the inner container can be transferred into the barrier and enclosed therein in a particularly simple way. The barrier may also be composed of nonwoven materials.

Particularly suitable materials for the porous barrier are plastics, glasses or ceramics. Advantageously the barrier is formed of polymers, in particular of copolymers or terpolymers. The polymers are, in particular, polyolefins. The porous barrier of the liquid dispenser is preferably formed from polymers based on at least one monomer from the group consisting of polyethylene, polypropylene, polystyrene, polyurethane, vinylidene chloride, vinylidene fluoride, tetrafluoroethylene, and hexafluoropropylene.

The inner container is normally composed of gastight and vaportight materials and is impervious for moisture. In one preferred construction the inner container is formed from a frangible material, in particular from glass. The wall thickness of the inner container is advantageously between about 100 and about 1000 μm, in particular between about 200 and about 500 μm, preferably about 300 μm. The inner container may be present in a variety of forms. For example, the inner container may be of cylindrical or tubular design and in particular may have a flattened end. The inner container may additionally be of syringelike or ampulelike design.

In another construction, which may also be actualized on its own, the inner container is formed from metals or composite materials, more particularly in the form of sheets. The metal is preferably aluminum. Preferably the aluminum is coated on the inner surface of the inner container with a material which is inert toward the curable liquid. Accordingly, premature curing of the liquid is prevented with particular advantage.

Composite materials suitable may in principle be all of the materials that are familiar to those skilled in the art. Composite materials with metals as components of the composite are preferred. Aluminum is a particularly suitable component of the composite, on account of its vaportight nature. The composite materials may in particular possess a laminar construction. The composite materials may in addition comprise sealing layers and/or adhesive layers, for example. With advantage the composite materials comprise what are called laminates, based in particular on plastics. Preference is given to composite materials based on metals, paper, plastics and/or surface-coating materials. The composite materials may be formed, for example, from the following components: polyethylene, polypropylene, polyethylene terephthalate, polyamide, polyvinyl chloride, surface-coating materials, paper and/or aluminum. Particular preference is given to the compositions set out below:
polyethylene-aluminum, -polyethylene terephthalate, polyvinyl chloride-aluminum-polyamide, polypropylene-aluminum-surface coating materials, polyethylene terephthalate-aluminum-polypropylene, and surface-coating materials-aluminum-polyethylene terephthalate-paper.

In one preferred structure the inner container has what is called a predetermined breakage point. A predetermined breakage point means a weak point in the inner container, at which, when external forces act on the liquid dispenser, damage, especially in the form of fractures, occurs with particular ease, thereby allowing the curable liquid to be released from the inner container. The predetermined breakage point may be, in particular, a constriction—for example, when the inner container is of glass and takes the form in particular of a glass ampule. Alternatively, the predetermined breakage point, preferably, may be a weak weld seam. This is especially advantageous when the inner container is formed of a composite material or of aluminum. The exertion of external forces on the liquid dispenser in this case produces rupture of the weld, seam, as a result of which the curable liquid is released from the inner container.

The outer container of the liquid dispenser is preferably formed of a flexible material, in particular of an elastic material. Suitable materials advantageously include plastics. The outer container may also be present in various three-dimensional design forms. For instance, the outer container may be, for example, a syringe, a cylinder, a flexible tube or an ampule, more particularly a twist-off ampule.

In one construction the application aid is designed in such a way that it is separate from the outer container. The application aid is preferably designed in such a way that it is connectable with the outer container. For the connection of the application aid and of the outer container, all of the connecting mechanisms familiar to those skilled in the art are suitable. Thus, for example, the application aid may possess an internal thread which is complementary to a corresponding external thread on the outer container. In this way the application aid and the outer container can be connected simply by being screwed together.

In another construction the application aid and the outer container are of one-piece design.

In one preferred construction the application aid has a needle-shaped element which points into the interior of the liquid dispenser, in particular into the interior of the outer container. The needle-shaped element is preferably of hollow-cylindrical design, in particular in the manner of a cannula. On connection of the application aid and the outer container, the inner container, an aluminum pouch for example, is perforated by the needle-shaped element of the application aid, with the formation, preferably, of an exit opening. With particular preference the needle-shaped element is the application aid itself, preferably in the form of a cannula. Therefore, with particular advantage, the needle-shaped element serves on the one hand to perforate the inner container and on the other hand as an application aid for the liquid that emerges after the inner container has been perforated. Consequently a liquid dispenser is provided which is comfortable and particularly simple in its handling.

It is likewise possible for the inner container to be perforated by a needle-shaped element only after successful connecting of the application aid and the outer container. For example, after the connecting of the application aid and the outer container, a needle may be passed through the hollow application aid in order to perforate the inner container.

In addition there may be sharp-edged means located in the interior of the outer container. The sharp-edged means may, for example, be saw teeth. The sharp-edged means are advantageously fixed on the inner surface of the outer container. As a result of exertion of pressure on the outer container, the inner container is at least partly torn open by the sharp-edged means, and so the liquid located within the inner container is able to emerge into the interior of the outer container.

In a further construction the liquid dispenser is in a pack, such as in a thermoformed or peel pack, for example. The pack may be formed in particular of a gastight material, such as of aluminum, for example. It is also possible for the pack to be formed of gastight composite materials, in particular with aluminum as a component of the composite. For further details regarding the composite materials, reference is made to the earlier description.

In one preferred construction the pack is formed at least partly of gas-permeable materials, and in particular materials impermeable to germs. Generally speaking, the materials are fiber web materials, based in particular on polyolefins. Preferred materials suitable are polyethylene and/or polypropylene, in particular high-pressure polyethylene and/or high-pressure polypropylene. Materials of this kind are available commercially, for example, under the names Tyvek® and TYPAR®.

The liquid dispenser is preferably sterilized, with preferably the outside of the liquid dispenser and, in particular, the outside of the inner container being sterile. The delivery opening of the application aid is advantageously open at least, during the sterilization of the liquid dispenser. In this way, sterilization of the interior of the outer container of the liquid dispenser is possible as well. Suitable methods of sterilization include, in particular, plasma, ethylene oxide, hydrogen peroxide or steam sterilization. For the purpose of sterilization, the liquid dispenser is transferred in general to a pack, which in particular has a sterilization opening. The sterilization opening may be, for example, an opening in the pack. For example, the pack may be open at one end. The sterilization opening is preferably a part of the wall of the pack, the sterilization opening being formed preferably of a gas-permeable material and, in particular a material impermeable to germs. Regarding the gastight material, which is in particular impermeable to germs, reference is made to the earlier description. The sterilization opening can advantageously be given a gastight closure. Suitable closure techniques, particularly after the end of sterilization of the liquid dispenser, include, for example, adhesive-bonding techniques and welding techniques. It is also possible for the sterilization opening to be closed with a gastight material, in particular in the form of a film. Where appropriate, prior to the closure of the sterilization opening, the liquid dispenser may be subjected to at least one drying step. In one particularly preferred embodiment the liquid dispenser is sterile and free from residual moisture.

The curable liquid in the inner container is preferably sterile. For example, the liquid may have been subjected to sterile filtration. The liquid may more particularly be an adhesive composition, based in particular on cyanoacrylate monomers. The cyanoacrylate monomers may be nonabsorbable or absorbable in the human and/or animal body. The cyanoacrylate monomers may additionally be present in the form of a mixture of different cyanoacrylate monomers. The adhesive composition preferably comprises alkyl cyanoacrylate monomers and/or alkoxy-alkyl cyanoacrylate monomers and/or alkyl ester cyanoacrylate monomers. The alkyl chains of the cyanoacrylate monomers have in particular a carbon number of between 1 to 12, in particular between 1 and 10. The alkyl cyanoacrylate monomers may be, for example, at least one monomer from the group encompassing ethyl, n-butyl, isobutyl, and n-octyl cyanoacrylate, preference being given to n-butyl and/or n-octyl cyanoacrylate. The alkoxy-alkyl cyanoacrylate monomers are, in particular, methoxypropyl and/or ethoxyethyl cyanoacrylate, preferably methoxypropyl cyanoacrylate. The alkyl ester cyanoacrylate monomers are selected preferably from the group encompassing butyllactoyl, butylglycoloyl, isopropylglycoloyl, ethyllactoyl, and ethylglycoloyl cyanoacrylate.

The curable liquid may comprise additional additives. The additives may in particular be plasticizers. Suitable plasticizers include in particular all of the compounds that are familiar to those skilled in the art. Examples of suitable plasticizers include fatty acid alkyl esters, especially isopropyl myristate, ethyl myristate, alkyl laurates, alkyl palmitates, diacetyl adipate and/or butyl stearate. The plasticizers may additionally be phthalic esters, examples being dioctyl phthalate, dibutyl phthalate and/or butyl benzyl phthalate. Suitable further plasticizers include, in particular, citric esters, examples being tri-n-butylcitrate, acetyl trihexylcitrate and/or n-butyl trihexylcitrate.

The liquid may also contain compounds which have the effect in particular of influencing the viscosity of the liquid. In one preferred form the liquid comprises polymers, in particular absorbable polymers. The polymers may in particular be copolymers and/or terpolymers, preferably based on at least one monomer from the group consisting of lactide, glycolide, caprolactone, trimethylene carbonate, p-dioxanone, and hydroxybutyric acid. With particular preference the liquid comprises a copolymer of lactide and caprolactone. The liquid may additionally comprise a terpolymer based on trimethylene carbonate, glycolide, and caprolactone. It may also be of advantage for the liquid to contain polydioxanone.

Particular provision is made for the liquid to comprise a mixture of cyanoacrylate monomers and polymers, preferably absorbable polymers. For further features of the cyanoacrylate monomers and also of the polymers, reference is made to the earlier description.

Moreover, the liquid may comprises stabilizers, especially for the purpose of preventing premature polymerization of curable components that are present in the liquid. The stabilizers preferably comprise at least one stabilizer from the group consisting of sulfur dioxide, phosphoric acid, hydroquinone, tert-butylhydroxyanisole, acetic acid, and sulfuric acid. The individual stabilizers of the liquid may be present in different amounts. The stabilizers may be present preferably in the following ranges of amounts: sulfur dioxide: about 20 to about 100 ppm, phosphoric acid: about 10 to about 500 ppm, hydroquinone: about 100 to about 3000 ppm, tert-butylhydroxyanisole: about 100 to about 3000 ppm, acetic acid: about 10 to about 500 ppm, and sulfuric acid: about 10 to about 500 ppm.

We additionally provide methods of producing the liquid dispenser for use in medicine, in particular for treating human and/or animal wounds. The wounds are preferably internal wounds. For further features and details of the liquid dispenser, reference is made to the earlier description.

Further features of our dispensers and methods will become apparent from the representative examples below, on the basis of figures. The figures are hereby made part of the description through express reference.

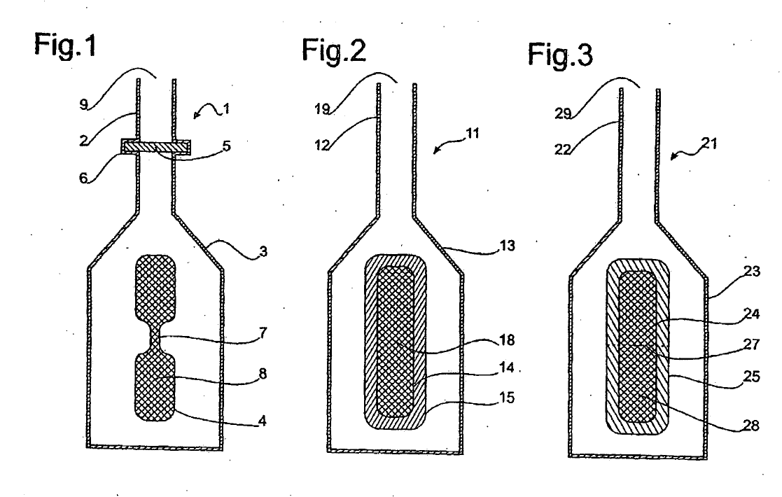

FIG. 1 shows a liquid dispenser 1 comprising a plastic ampule 3 which is designed as an outer container and has a cannulalike application aid 2 and, located therein and designed as a separate inner container, a glass ampule 4. Located within the application aid 2 is an open-pore polyethylene foam 5 designed as a barrier. On its inner surface the application aid 2 has fixing means in the form of bulges 6. The polyethylene foam 5 is locked in the bulges 6, as a result of which the polyethylene foam 5 is secured, for example, against lateral slipping, particularly in the course of the proper use of the liquid dispenser 1. The glass ampule 4 has a predetermined breakage point in the form of a constriction 7. As a result of exertion of pressure on the plastic ampule 3, the glass ampule 4 ruptures in the region of the constriction 7, as a result of which a liquid adhesive composition 8 which is present as a curable liquid therein and is based on n-butyl cyanoacrylate monomers emerges from the glass ampule 4 into the interior of the plastic ampule 3. As a result of the proper use of the liquid dispenser 1, the released adhesive composition 8 flows through the polyethylene foam 5 into the application aid 2 and ultimately, through the delivery opening 9, which has a clear cross section of about 0.8 mm², out of the liquid dispenser 1. The polyethylene foam 5 exerts a filter function and retains the glass splinters formed as a result of the breaking of the glass ampule. This enables trouble-free application of the adhesive composition 8 to a wound that is to be treated.

FIG. 2 shows a liquid dispenser 11 comprising a plastic ampule 13, which is designed as an outer container and has a cannulalike application aid 12, and, located therein and designed as a separate inner container, a glass ampule 14. The glass ampule 14 is completely enveloped by a shrink-on tube 15, designed as a porous barrier, and contains a curable liquid in the form of an adhesive composition 18 based on n-butyl and n-octyl cyanoacrylate monomers. When force is exerted on the plastic ampule 13, the glass ampule 14, whose wall thickness is about 300 μm, is destroyed, and the adhesive composition 18 emerges as a result. Through the porous shrink-on tube 15, the adhesive composition 18 passes into the interior of the plastic ampule 13, while the glass splinters are retained by the porous shrink-on tube 15. As a result of the proper use of the liquid dispenser 11, the adhesive composition 18 can be applied to the wound region through the delivery opening 19 of the application aid 12, which has a clear cross section of about 0.8 mm².

FIG. 3 shows a liquid dispenser 21 comprising a plastic ampule 23, which is designed as an outer container and has a cannulalike application aid 22, and, an aluminum pouch 24, which is designed as a separate inner container and has, located therein, a curable liquid in the form of an adhesive composition 28 based on alkoxy-alkyl cyanoacrylate and alkyl ester cyanoacrylate monomers. The aluminum pouch 24 has a predetermined breakage point in the form of a weak weld seam 27. As a result of external action of force on the plastic ampule 23, the aluminum pouch 24 bursts in the region of the weak weld seam 27, and, as a result, the adhesive composition 28 passes into the interior of the plastic ampule 23. Through the proper use of the liquid dispenser 21, the emergent adhesive composition 28 can be applied to the wound to be treated, through the delivery opening 29 with a clear cross section of about 0.8 mm².

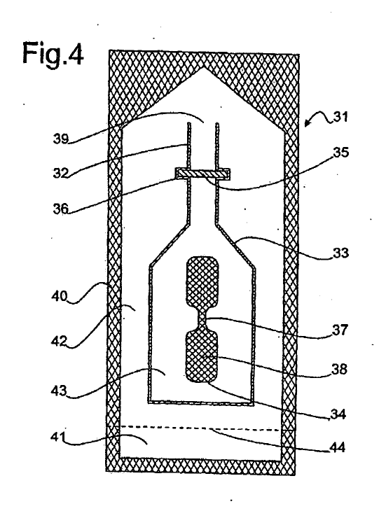

FIG. 4 shows a liquid dispenser 31 comprising a plastic ampule 33, which is designed as an outer container and has a cannulalike application aid 32, and a glass ampule 34, which is designed as a separate inner container and has a predetermined breakage point in the form of a constriction 37. Located within the glass ampule 34, as a curable liquid, is an adhesive composition 38 based on alkyl and alkoxyalkyl cyanoacrylate monomers. Located within the application aid 32 there is an open-pore polyethylene foam 35, which is designed as a barrier and is locked by fixing means, in the form of bulges 36, on the inner surface of the application aid 32. The liquid donor 31 is located within an aluminum pouch 40, designed as a pack, which has a TYVEK® window 41, designed as a sterilization opening, integrated at one end. In the course of the sterilization procedure, the sterilizing gas passes through the TYVEK® window 41 into the interior 42 of the aluminum pouch 40. Through the open delivery opening 39 and the open-pore polyethylene foam 35, the sterilizing gas passes, in addition, into the interior 43 of the plastic ampule 33. After the end of sterilization, the aluminum pouch 40 is welded through along the border 44, with removal of the TYVEK® window 41, and so the liquid dispenser 31 is enclosed vapor tightly in the aluminum pouch 40, the interior 42 of the aluminum pouch 40 and especially the interior 43 of the plastic ampule 33 being sterile.

As a result of the spatial separation of the delivery opening 9, 19, 29, and 39 from the barrier 5, 15, and 35, and also from the inner container 4, 14, 24, and 34, it is possible for the delivery opening 9, 19, 29, and 39 to be given an arbitrary configuration or form, such as a circular or flat nozzle, for example. As a result, adaptation to the desired delivery mode of the curable liquid 8, 18, 28, and 38 is possible. The delivery opening 9, 19, 29, and 39 is preferably open. Accordingly, in a particularly advantageous way, the liquid dispenser 1, 11, 21, and 31 can be sterilized following transfer to a suitable pack 40. Prior to the gastight welding of the pack 40, drying steps may be interposed. In this way it is possible to provide liquid dispensers 1, 11, 21, and 31 which are sterile and, in particular, are dry, i.e., are free from residual moisture. The inner container 4, 14, 24, and 34 is preferably welded into the outer container 3, 13, 23, and 33.

1-26. (canceled)

27. A liquid dispenser for medical purposes in the form of an applicator, having an outer container, which comprises a hollow application aid made from a nonporous material, in the form of a cannula, and having an inner, vaportight, and closed container with a curable liquid located therein, the application aid having a delivery opening with a clear cross section of between about 0.1 and about 5 mm².

28. The liquid dispenser of claim 27, wherein the delivery opening has a clear cross section of between about 0.2 and about 3.0 mm².

29. The liquid dispenser of claim 27, further comprising a porous barrier which is pervious to the liquid and impervious for fragments of the inner container located between the inner container and the delivery opening of the application aid.

30. The liquid dispenser of claim 29, wherein the porous barrier possesses pores having a diameter of between about 0.1 and about 500 μm.

31. The liquid dispenser of claim 29, wherein the porous barrier is designed as a porous membrane or porous foam in the form of a filter.

32. The liquid dispenser of claim 29, wherein the application aid has, on an inner surface, at least one fastener for the porous barrier.

33. The liquid dispenser of claim 32, wherein the at least one fastener is/are bulge(s) in the inner surface.

34. The liquid dispenser of claim 29, wherein the porous barrier envelops the inner container.

35. The liquid dispenser of claim 29, wherein the porous barrier is a liquid-pervious envelope for the inner container, shaped as a liquid-pervious flexible tube.

36. The liquid dispenser of claim 29, wherein the porous barrier is formed from a polymer, based on at least one monomer selected from the group consisting of polyethylene, polypropylene, polystyrene, polyurethane, vinylidene chloride, vinylidene fluoride, tetrafluoroethylene, and hexafluoropropylene.

37. The liquid dispenser of claim 27, wherein the inner container is formed from a frangible glass material.

38. The liquid dispenser of claim 27, wherein the inner container is formed from aluminum or from composite materials with aluminum as a component of the composite.

39. The liquid dispenser of claim 38, wherein the aluminum is coated on an inner surface of the inner container with a material which is inert toward the curable liquid.

40. The liquid dispenser of claim 27, wherein the inner container has a predetermined breakage point in the form of a constriction or a partable weld seam.

41. The liquid dispenser of claim 27, wherein the application aid is separate from the outer container and connectable with the outer container.

42. The liquid dispenser of claim 27, wherein the application aid and the outer container are one-piece.

43. The liquid dispenser of claim 27, wherein the application aid has a needle-shaped element in the form of a cannula which points into an interior portion of the liquid dispenser.

44. The liquid dispenser of claim 27, wherein the liquid dispenser is in a thermoformed or peel pack.

45. The liquid dispenser of claim 44, wherein the pack is formed at least partly of a gas-permeable material impermeable to germs.

46. The liquid dispenser of claim 27, wherein the liquid dispenser is sterilized.

47. The liquid dispenser of claim 27, wherein the curable liquid is sterile.

48. The liquid dispenser of claim 27, wherein the curable liquid is an adhesive composition, based on cyanoacrylate monomers.

49. The liquid dispenser of claim 48, wherein the cyanoacrylate monomers are n-butyl and/or n-octyl cyanoacrylate monomers.

50. The liquid dispenser of claim 48, wherein the cyanoacrylate monomers are alkoxy-alkyl and/or alkyl ester cyanoacrylate monomers.

51. The liquid dispenser of claim 27, wherein the liquid comprises absorbable polymers.

52. A method of treating human and/or animal wounds comprising contacting a liquid dispensed from the liquid dispenser of claim 27 with a human and/or animal wound.
